SEQUENCE LISTING (10 sequences)

SEQ ID NO: 1 - DNA, 4 bp, Artificial Sequence
  SYNTHETIC
  misc_feature (1)..(4): n is a, c, g, or t
nnnn

SEQ ID NO: 2 - DNA, 8 bp, Artificial Sequence
  Synthetic
acgtgtaa

SEQ ID NO: 3 - DNA, 8 bp, Artificial Sequence
  Synthetic
ttacacgt

SEQ ID NO: 4 - DNA, 8 bp, Artificial Sequence
  Synthetic
tggcttat

SEQ ID NO: 5 - DNA, 8 bp, Artificial Sequence
  Synthetic
ataagcca

SEQ ID NO: 6 - DNA, 16 bp, Artificial Sequence
  Synthetic
acgtgtaatggcttat

SEQ ID NO: 7 - DNA, 16 bp, Artificial Sequence
  Synthetic
ataagccattacacgt

SEQ ID NO: 8 - DNA, 16 bp, Artificial Sequence
  Synthetic
acgtgtaattacacgt

SEQ ID NO: 9 - DNA, 12 bp, Artificial Sequence
  Synthetic
  misc_feature (9)..(12): n is a, c, g, or t
acgtgtaannnn

SEQ ID NO: 10 - DNA, 12 bp, Artificial Sequence
  Synthetic
  misc_feature (1)..(4): n is a, c, g, or t
nnnnttacacgt

(19) United States
(12) Patent Application Publication
(10) Pub. No.: US 2022/0119805 A1
(43) Pub. Date: Apr. 21, 2022

(54) Methods of Suppressing Adaptor Dimer Formation in Deep Sequencing Library Preparation

(71) Applicant: Icahn School of Medicine at Mount Sinai, New York, NY (US)

(72) Inventor: Ravi Sachidanandam, Brooklyn, NY (US)

(73) Assignee: Icahn School of Medicine at Mount Sinai, New York, NY (US)

(21) Appl. No.: 16/625,417

(22) PCT Filed: Jun. 27, 2018
(86) PCT No.: PCT/US2018/039771

§ 371 Date: Dec. 20, 2019

Related U.S. Application Data
(60) Provisional application No. 62/525,437, filed on Jun. 27, 2017.

(51) Int. Cl. C12N 15/10; C12Q 1/6869; C12Q 1/6855

(52) U.S. Cl. CPC C12N 15/1082; C12Q 1/6855; C12Q 1/6869

(57) ABSTRACT

Disclosed are methods of suppressing adaptor dimer formation comprising: providing a target polynucleotide with a 5′ and 3′ end; providing a double stranded DNA adaptor with a 5′ end and a 3′ end that have sequence complementary to each other, ligating the double stranded adaptor to the target polynucleotide to form a ligation product. Also provided is a method of preparing a library of nucleic acid sequences comprising: providing a double-stranded DNA adaptor with 5′ and 3′ ends having a sequence complementary to each other, contacting the adaptor with a target nucleic acid sequences having a 5′ and a 3′ end, and ligating the adaptor with complementary sequence to the 5′ and 3′ ends of the target nucleic acid sequence using a double stranded DNA ligase. The disclosure also provides kits for suppression of adaptor dimer formation in deep sequencing containing a double stranded DNA adaptor with 5′ and 3′ ends having a sequence complementary to each other, suitable enzymes, buffers, dNTPS, etc.

### RELATED APPLICATIONS

This application is the national phase entry of PCT/US2018/039771, filed Jun. 27, 2018 and claims priority to U.S. Provisional Application No. 62/525,437, filed on Jun. 27, 2017, entitled Methods of Suppressing Adaptor Dimer Formation in Deep Sequencing Library Preparation, which is incorporated herein in its entirety.

### FIELD OF INVENTION

The present disclosure relates generally to methods for preparing a library for sequencing, which involve addition of adaptors on both ends of target polynucleotides. More specifically, the present disclosure relates to adaptor dimers and a method of preparing a library of template polynucleotides that suppresses or prevents the formation or abundance of adaptor dimers.

### REFERENCE TO A SEQUENCE LISTING

This application contains a sequence listing. It has been submitted electronically and was created as an ASCII text file entitled 46574-5_ST25.txt on Nov. 17, 2021 and is 2,421 bytes in size.

### BACKGROUND

In most sequencing-by-synthesis platforms, the product that is loaded on the sequencer consists of target single stranded DNA fragments (usually <1 kb long) flanked by platform-specific “adaptors” on both ends. These adaptors can be single stranded or double stranded nucleotide sequences (either RNA or DNA). The adaptors serve as primers during universal PCR amplification or as initiators during sequencing by synthesis. The adaptors are typically added to the inserts through ligation prior to the sequencing process. An undesirable consequence of this reaction is the formation of dimers consisting of the 3′ adaptor and the 5′ adaptor with no insert sequence, which in subsequent reactions involving cloning or amplification gives rise to significant background noise. Such occurrence of adaptor dimers not only consumes valuable sequencing space; it also distorts the quantification of transcripts in RNA sequencing experiments. Thus, reducing the abundance is the focus of many techniques used to clean up the final libraries loaded on the sequencer.

Usual strategies for adaptor dimer suppression include size selection using gels or AMPure beads available from Beckman Coulter to remove the adaptor dimers. These strategies however are not foolproof, as seen from the occurrence of adaptor dimers in RNA sequencing libraries and are quite leaky when the inserts are particularly short, as in small RNA sequencing. Other known strategies have involved the use of constructs that bind to the adaptor-dimer junction to block PCR amplification.

### SUMMARY OF THE INVENTION

The present disclosure provides an efficient method of suppressing the occurrence and abundance of dimer formation in a deep sequencing library that is sensitive, quick and accurate without the need for additional strategies.

In one embodiment, the present disclosure provides a method for suppressing or preventing adaptor dimer formation characterized by the steps of: providing a target polynucleotide with a 5′ end and a 3′ end; providing at least two adaptors with ends having nucleotide sequence that is complementary to each other, ligating the adaptors to the target polynucleotide to form a ligation product. The two adaptors disclosed herein can be a double stranded DNA adaptor or a single stranded RNA and/or a single stranded DNA adaptor. The target polynucleotide may be a double stranded DNA or a complementary DNA. The ligation product is the target polynucleotide with the adaptor ends having a complementary sequence flanking on each end of the target. The ends of the disclosed adaptors may be a 4-mer or 6-mer or an 8-mer and is capable of suppressing the adaptor dimer formation by more than about 90%. The method may further include a double stranded DNA ligase or a single stranded RNA ligase and may require no addition of a hairpin oligonucleotide to the ligation reaction.

In another embodiment, the present disclosure provides a method of preparing a library of nucleic acid sequences. The method comprising the steps of: providing at least two adaptors with ends having nucleotide sequence that is complementary to each other, contacting the adaptor with a target nucleic acid sequences having a 5′ and a 3′ end, and ligating the adaptor ends with complementary sequence to the 5′ and 3′ ends of the target nucleic acid sequence using a double stranded DNA ligase or single stranded RNA ligase. The adaptor ends flanking the target nucleic acid sequence is configured to suppress the formation or abundance of adaptor dimers. The two adaptors disclosed herein can be a double stranded DNA adaptor or a single stranded RNA and/or a single stranded DNA adaptor. The target polynucleotide may be a double stranded DNA or a complementary DNA.

In another embodiment, the present disclosure provides a method for suppressing or preventing adaptor dimer formation in SMART sequencing characterized by the steps of: providing a target polynucleotide with a 5′ end and a 3′ end; providing at least two adaptors with ends having nucleotide sequence that is complementary to each other, adding the adaptors to the target polynucleotide in a ligation free reaction. The target polynucleotide may be a complementary DNA. The method may further comprise addition of reverse transcriptase to facilitate the synthesis of complementary DNA. The method may also comprise the addition of a first strand synthesis primer and a template switching primer.

In another embodiment the present disclosure provides a kit for suppression of adaptor dimer formation comprising at least two adaptors with ligating ends having nucleotide sequence that is complementary to each other. The adaptors in the kit may be a double stranded DNA adaptor or a single stranded RNA or DNA adaptor or both. The adaptors disclosed herein may at least be a 4-mer sequence. The kit may further comprise enzymes such as ligase or polymerase.

### DESCRIPTION OF DRAWINGS

FIG. 1 is a schematic representation of a double stranded adaptor for use in DNA sequencing. 1A shows the universal adaptor design with ligating ends barcode A (5′ ACGTGTAA 3′ (SEQ ID NO: 2) and complimentary 5′ TTACACGT 3′(SEQ ID NO: 3)) and barcode B (5′ TGGCTTAT 3′ (SEQ ID NO: 4) and complimentary 5′ ATAAGCCA 3′(SEQ ID NO: 5)) that are non-complementary to each other flanking an insert. 1B shows the formation of adaptor dimers that lack the inserts when the adaptor design of 1A is used (5′ ACGTGTAATGGCTTAT 3′ (SEQ ID NO: 6) and 5′ ATAAGCCATTACACGT 3′(SEQ ID NO: 7)). 1C shows the adaptor designs of the present disclosure with ligating ends that are complementary to each other barcodes 1 (SEQ ID NO: 2 and complimentary SEQ ID NO: 3) and barcode 2 (SEQ ID NO: 3) and complimentary (SEQ ID NO: 2). 1D and 1E shows formation of adaptor dimer (5′ ACGTGTAATTACACGT 3′ (SEQ ID NO: 8)) with the adaptor design of 1C.

FIG. 2 shows a schematic representation of single stranded adaptor for use in RNA-sequence (especially small RNA sequence). 2A shows the universal adaptor design with ligating ends barcode 1 and barcode 2 that are non-complementary to each other. 2B shows the adaptor design with ligating ends having a complementary sequence (5′ ACGTGTAANNNN 3′ (SEQ ID NO: 9) and 5′ NNNNTTACACGT 3′ (SEQ ID NO: 10) in addition to the N's that flank an insert.

FIG. 3 is a graphic representation of the suppression of adaptor dimers using the adaptors with complementary ligating ends. The matrix plot depicts pairs of barcodes in adaptor dimers. The matrix uses a color plot to show deviations from the mean, or expected values if the barcode pairs randomly assorted. There are 96 barcodes on each side, leading to 9216 combinations of barcode pairs. The 96 rows represent barcodes on the left while the 96 columns are barcode on the right, as defined in FIG. 1C. When the barcodes are identical on both sides (the diagonal), there is almost perfect suppression, shown by the dark shade used to mark zeroes, or lack of insert. The geometry in the case of identical adaptors on both sides is shown in FIG. 1C, which demonstrates that having complementary sequences at the ends of the adaptor leads to the suppression of adapter-dimers in the sequencing library.

FIG. 4 is a graphic representation of adaptor ends having a complementary sequence that do not suppress a product with an insert. The matrix shows the combinations of barcodes for the most abundant insert, rows are barcode 1 and columns are barcode 2, as in FIG. 1. The absence of suppression along the diagonal in this plot is a reflection of the fast that complementary ends of the adaptors do not suppress reads with normal inserts between them.

FIG. 5 is a schematic representation of the reproducibility of the results. The data here is for the most abundant insert in the mRNA sequence dataset (from a fragment of the gene ssrA of E. coli). The scatter plots show; panel A) That the 5′ adaptors (A) are consistent between replicas G (y-axis) and W (x-axis), panel B) The 3′ adaptors (B) are consistent between replicas G (y-axis) and W (x-axis). In contrast, the barcodes of 5′ adaptor (A) and 3′ adaptor (B) shows scatter (Panel C for sample G), and Panel D for sample W) demonstrating the results in Panel A and B are not artifacts.

FIG. 6 shows a schematic representation of using adaptor with complementary ends to suppress adaptor dimers in SMART-Sequencing. The left panel 6A shows the standard method of preparing SMART-Sequence libraries, which result in adaptor dimers. The adaptor of the present disclosure (SEQ ID NO: 2 and 3) may be used, as shown in the right panel 6B, to reduce or prevent the formation of adaptor dimers.

### DETAILED DESCRIPTION

It is an object of the present disclosure to provide a method for suppressing the formation of adaptor dimers in deep sequencing library preparation.

The disclosed method may provide a target polynucleotide with a 5′ and a 3′ end. As used herein, the term “target polynucleotide” refers to a nucleic acid molecule to which adaptors are ligated on both 5′ and 3′ ends of the target. The target nucleic acid may be any molecule that may be amplified or sequenced and may be obtained from any biological source by use of well-known methods. The biological samples may be obtained from any subject, human or non-human or from any cell lines that may be fresh or fixed. The target nucleic acid may be any length suitable for use in the methods of the present disclosure. For example, the target nucleotides may be about 10 nucleotides to about 1000 or about 1500 nucleotides in length or longer. The target polynucleotide may be a double stranded DNA or a complementary DNA or cDNA. The polynucleotide may also be a single stranded RNA.

The disclosed method may further include the addition of at least two adaptors with ligating ends having sequence complementary to each other. The adaptors of this disclosure may be a double stranded DNA adaptor or it may be a single stranded RNA or DNA adaptor. The double stranded DNA or single stranded RNA or DNA adaptor disclosed herein, may refer to any oligomer or oligonucleotide of varying length and characterized by ligating ends having nucleotide sequence or codes that is complementary to each other.

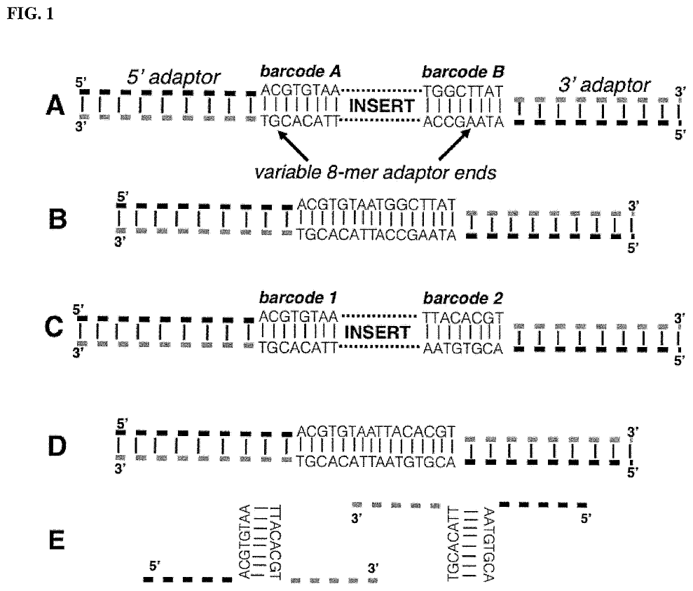

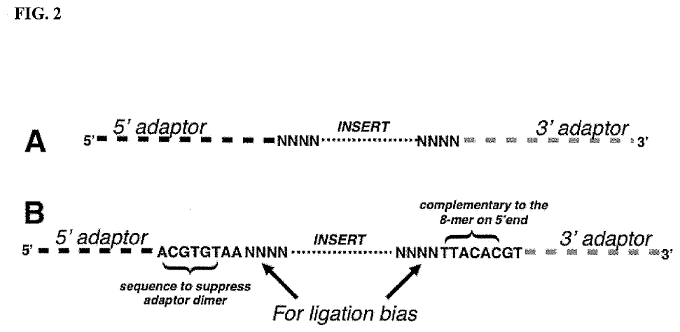

A universal double stranded DNA or a single stranded RNA adaptor design, which are currently in use, is shown in FIGS. 1A and 2A, respectively. These universal adaptors are known to have ligating ends that are non-complementary to each other. For example, as shown in FIG. 1A, the 5′ end of the first adaptor or “Barcode A” has a complementary 3′ strand. Similarly, the 5′ end of the second adaptor or “Barcode B” has a complementary 3′ strand. But the ligating ends of Barcodes A and B, which flank the insert, have sequence that are non-complementary to each other. The ligating ends of a universal single stranded RNA adaptor may also include randomized codes, such as for example, a 4-mer N's), wherein the N may be any one of the four nucleotides A, T, G and C and are used primarily to reduce the ligation bias (FIG. 2A).

But for the suppression of the adaptor dimer formation disclosed herein, the sequence of the double stranded adaptor ligating ends or single stranded RNA ligating ends may be complementary to each other, as shown in FIG. 1C and FIGS. 2B and C respectively. For example, as shown in FIG. 1C, the 5′end and the 3′end of the Barcode 1 is complementary to each other. Similarly, the 5′end and the 3′end of Barcode 2 are complementary to each other. But unlike the universal adaptors shown in FIGS. 1A, 1B and FIG. 2A, the method disclosed herein may require that the ligating ends of both Barcode 1 and Barcode 2 are also complementary to each other, as shown in FIGS. 1C and 2C, respectively.

Similarly, a universal single stranded RNA may include adaptors with ends that are non-complementary to each other. For example, as shown in FIG. 2A, the insert is flanked by random N's on either side and the sequences of these adaptors are non-complementary to each other. But the ligating ends of the insert shown in FIG. 2B has adaptor ends that have complementary sequence to each other. In addition to the adaptor ligating ends having a complementary sequence, a single stranded RNA adaptor disclosed herein, may optionally include randomized N's, as shown in FIG. 2B, to reduce the ligation bias.

The ligating adaptor ends with a complementary sequence, as disclosed in the present disclosure, may at least be 4-mer in length. The adaptor ends may also be at least 6-mer in length, or at least 8-mer in length or at least 10-mer in length or at least 15-mer in length or at least up to 25-mer in length or about 30-mer in length or longer. The advantage of using the strategy of complementary ligating ends on the adaptors in the present disclosure is that no additional strategies such as adding end blockers or enzymatic adenylation of adaptor is required to suppress the formation of adaptor dimers.

The disclosed method may also include the step of ligating the adaptor ends to the target polynucleotide to form a ligation product. Accordingly, the ligation product may be characterized by the target polynucleotide flanked by the adaptor ends of the present disclosure (adaptor end-target-adaptor end) with a complementary sequence. The ligation reaction may be catalyzed by a double stranded DNA ligase. The ligation reaction may also be catalyzed by a single stranded RNA ligase when the target nucleotide is a single stranded RNA. Besides the double stranded adaptor having a complementary sequence, the disclosed method requires no addition of any hairpin oligonucleotides to block the adaptor dimer. The disclosed method may suppress the adaptor dimer formation by more than about 20%, or more than about 40%, or more than about 60%, or more than about 70% or more than about 80% or more than about 90% or greater, compared to any conventional method such as but not limited to those which either use no adaptors or rely on addition of hairpin oligonucleotides to suppress the adaptor dimer formation.

In another embodiment, the present disclosure provides a method for preparing a library of nucleic acid sequences. The method includes the step of: providing at least two adaptors with ligating ends having nucleotide sequence that is complementary to each other. The adaptors may be a double stranded DNA adaptor or a single stranded RNA adaptor. The adaptors disclosed herein, refers to any oligomer or oligonucleotide characterized with ends having a nucleotide sequence complementary to each other that flanks the ends of a target nucleotide. A typical or universal double stranded DNA or a single stranded RNA adaptor design, which are currently in use, is shown in FIGS. 1A and 2A respectively. These universal adaptors are known to have ligating ends that are non-complementary to each other. For example, as shown in FIG. 1A, the 5′ end of the first adaptor or Barcode A has a complementary 3′ strand. Similarly, the 5′end of the second adaptor or Barcode B has a complementary 3′ strand. But the ligating ends of Barcode A and B have sequence that are non-complementary to each other. The ligating ends of a universal single stranded RNA adaptor may also include randomized codes, such as for example, a 4-mer N's (NNNN), wherein the N may be any one of the four nucleotides A, T, G and C and are used to reduce the ligation bias (FIG. 2A).

But for the suppression of the adaptor dimer formation disclosed herein, the sequence of the double stranded adaptor ligating ends or single stranded RNA ligating ends may be complementary to each other, as shown in FIG. 1C and FIGS. 2B and 2C respectively. For example, as shown in FIG. 1C, the 5′end and the 3′end of the Barcode 1 is complementary to each other. Similarly, the 5′end and the 3′end of Barcode 2 are complementary to each other. But unlike the universal adaptors shown in FIGS. 1A, 1B and FIG. 2A, the method disclosed herein may require that the ligating ends of both Barcode 1 and Barcode 2 are also complementary to each other, as shown in FIGS. 1C and 2C, respectively.

Similarly, a universal single stranded RNA may include adaptors with ends that are non-complementary to each other. For example, as shown in FIG. 2A, the insert is flanked by random N's on either side and the sequences of these adaptors are non-complementary to each other. But the ligating ends of the insert shown in FIG. 2B has adaptor ends that have complementary sequence to each other. In addition to the adaptor ligating ends having a complementary sequence, a single stranded RNA adaptor disclosed herein, may optionally include randomized N's, as shown in FIG. 2B, to reduce ligation bias.

The ligating adaptor ends with a complementary sequence, as disclosed in the present disclosure, may at least be 4-mer in length. The adaptor ends may also be at least 6-mer in length, or at least 8-mer in length or at least 10-mer in length or at least 15-mer in length or at least up to 25-mer in length or about 30-mer in length or longer. The advantage of using the strategy of complementary ligating ends on the adaptors in the present disclosure is that no additional strategies such as adding end blockers or enzymatic adenylation of adaptor is required to suppress the formation of adaptor dimers.

The disclosed method may also include the step of contacting the adaptor with a target nucleic acid sequence having a 5′ and 3′ end and ligating the adaptor to the 5′ and 3′ ends of the target nucleic acid in the presence of a double stranded DNA ligase. The ligation reaction may also be catalyzed by a single stranded RNA ligase when the target nucleotide is a single stranded RNA. The ligation of the adaptor and target nucleotides may be accomplished using a variety of standard techniques available and well established. The resulting ligation products or adaptor-target-adaptor library can then be used for PCR amplification or preparation of a library of nucleic acid sequences.

The present disclosure also includes a method for suppressing or preventing adaptor dimer formation in deep sequencing libraries that are made using single stranded universal oligonucleotides such as SMART (Switching Mechanism at 5′ End of RNA Template) technology. The ligase free methodology of SMART may add universal adaptors directly to both ends of the first-strand cDNA by using the template switching activity of reverse transcriptases (Chenchik et al. 1998). Two primers may be used in the reaction, a first strand synthesis primer and a template switching primers. Often times these primers bind together and extend forming adaptor dimers as shown in FIG. 6A. By adding a complementary sequence on each of these primers this adaptor-dimer formation can be prevented, by blocking its amplification, as shown in FIG. 6B.

In yet another embodiment the present disclosure provides a kit for reducing adaptor dimer formation comprising: a double or single stranded oligonucleotide adaptor with parts that are complementary in sequence to each other. The adaptors may be added via ligation of template switching mechanisms. The adaptors disclosed herein may at least be a 4-mer or at least a 6-mer or at least an 8-mer or at least a 10-mer or at least a 15-mer or about 30-mer in length or longer. The kit may include adaptors with ends that are either of same length, for example, a 8-mer or different lengths. The kit may also include suitable primers of appropriate nucleotide sequence for use with the adaptors. The kits may additionally comprise buffers, enzymes, such as for example, a DNA or RNA ligase or polymerase, dNTPs, and the like.

The method of the present disclosure will be described in further detail with reference to the following embodiments, for the purpose of making the objectives, technical solutions and advantages of the present invention clearer. It shall be understood that the specific embodiments described herein are illustrative only for the invention and not intended to limit the scope of the invention.

### EXAMPLES

### Example 1: Isolation of Total RNA from E. coli and rRNA Removal

In order to study the suppression of the adaptor dimers, total RNA from E. coli was first isolated using standard procedures. Then 1 μg of total RNA was used as input for rRNA removal.

The rRNA removal procedure involved addition of 225 μl of Ampure Beads in a 1.5 ml microcentrifuge tube containing the total RNA and placing the tube on a magnetic stand with the cap open for one minute. The resulting supernatant was discarded and the beads were washed with 2250 RNAse free water. After the liquid was discarded, 650 of magnetic bead resuspension solution was added and vortexed to resuspend the beads. To this 1 μl of Riboguard RNAse inhibitor was added and mixed using a pipette and set aside at room temperature. Then 8 μl of Ribo-zero solution containing probes was added to the mix to hybridize the probes to rRNA present in the sample. The tube containing the mix was then placed on a preheated heat block or thermal cycler at 68° C. and incubated for 10 minutes. After the tube was removed from the heat block, it was centrifuged briefly and incubated again at room temperature for 5 minutes. The removal of rRNA from the sample was then accomplished by combining the probe-hybridized samples with washed magnetic beads and incubating at room temperature for 5 minutes. The tube was placed on the preheated heat block at 50° C. and incubated for another 5 minutes. The tube was then placed on a magnetic stand with cap open for another minute or until the mix was completely clear. From this 80-90 μl supernatant containing depleted RNA was transferred to a fresh 1.5 ml tube and set aside on ice. To this mix RNAse free water was added to bring the volume to 180 μl. Then 18 ul 3M sodium acetate, 2 μl of glycoblue was added and mixed by vortexing. Subsequently, 600 μl of 100% ethanol was added and mixed. The tube was set aside at −25° C. to −15° C. for at least an hour and centrifuged at 10,000 g for 30 minutes at 4° C. The resulting supernatant was then discarded and the precipitate was washed twice with 200 μl of freshly prepared 70% ethanol. The solution was centrifuged again to collect any residual supernatant. The final pellet was then dissolved in 14 μl RNAse free water. The recovered RNA sample was now depleted of rRNA.

### Library Preparation

14 μl of rRNA free sample was then combined with 14 μl of RNA fragmentation buffer in a fresh microcentrifuge tube or plate and mixed well by pipetting. This step resulted in fragmented RNA. The tube was then heated for 10 minutes at 95° C. and then placed immediately on ice. To this 1 μl of NEXTflex™ First strand synthesis primer was added, heated again at 65° C. for 5 minutes and placed immediately on ice. Then a first strand synthesis enzyme mix was prepared by adding 1 μl of SuperScript R III Reverse Transcriptase per reaction to 4 μl of NEXTflex™ First strand buffer mix, mixed gently and centrifuged. Then, 20 μl of solution containing fragmented RNA, first strand synthesis buffer and 5 μl of first strand synthesis mix was combined to form a 25 μl volume mix. The tube containing the mix was then incubated sequentially at 25° C. for 10 minutes, at 50° C. for 50 minutes and 75° C. for 15 minutes. To prepare the second strand synthesis, 25 μl of first strand synthesis product was combined with 25 μl of second strand synthesis mix to form a 50 μl volume mix. This was mixed and incubated at 16° C. for 60 minutes. To this 90 μl of well-mixed AMPure XP beads was added, mixed well and incubated for 5 minutes at room temperature. The supernatant was then discarded without disturbing the beads. To the beads, 200 μl of freshly prepared 80% ethanol was added and incubated at room temperature for 30 seconds. The resulting supernatant was discarded and the beads were washed again twice. The final pellets were dried and resuspended in 41 μl of resuspension buffer mix. After the beads were rehydrated, the resuspended beads were incubated at room temperature for 2 minutes, placed on the magnetic stand for 5 minutes at room temperature until the supernatant was clear. 40 μl of the clear supernatant representing the complementary double stranded DNA insert or target, was then transferred to a fresh tube for the next steps involving end repair and ligation with adaptors.

### End Repair of Target DNA Template

40 μl of the second strand synthesis DNA was then mixed with 7 μl NEXTflex™ End Repair buffer mix and 30 of NEXTflex™ End Repair enzyme mix to form a 50 μl volume solution. This was incubated on a thermocycler at 22° C. for 30 minutes. To this 80 μl of well mixed AMPure XP beads was added and mixed by pipetting. The mix was incubated for 5 minutes at room temperature and then placed on the magnetic stand for 5 minutes or until the supernatant was clear. The supernatant was then removed and the beads were washed with 200 μl of freshly prepared 80% ethanol for at least 30 seconds at room temperature. The above step was repeated and the beads were washed at least twice with ethanol. The resulting beads were dried at room temperature for 5 minutes and resuspended in 170 re-suspension buffer. The beads were then carefully rehydrated and resuspended at room temperature for 2 minutes, placed again the magnetic stand for 5 minutes or until the supernatant was completely clear. From this 16 μl of clear supernatant, containing the end-repaired double stranded DNA, was transferred to a fresh well or microcentrifuge tube.

### Adaptor Ligation

20.5 μl of the above mentioned end repaired DNA solution was then mixed with 27.5 μl of NEXTflex™ Ligation mix and 2 μl of adaptor with ends having a complementary sequence to form a 50 μl volume mix. The adaptors used in this reaction were designed to have 96 distinct ends that were coded with 8-mers. An example of the geometry of this adaptor ligation is shown in FIGS. 1C and 1D. Because the adaptor ends have complementary sequences there are 9216 (96×96) possible combinations of adaptor ends. The benefit of using a defined set instead of a set of 4-mer N's at the end (256 different adaptors), is that the composition of the mixture is well-defined, making it easier to track the identities of molecules, thereby generating more confidence in the data and statistical inferences.

For controls, the end repaired DNA solution was first adenylated by combining 16 μl of end repaired DNA solution and 4.5 μl adenylated mix to form a 20.5 μl volume mix and incubated sequentially at 37° C. for 30 minutes and 70° C. for 5 minutes.

The mix containing the adaptors of the present disclosure or adenylated mix was then mixed with 40 μl AMPure XP beads, mixed and incubated on the magnetic plate or stand for 5 minutes at room temperature or until the supernatant was completely clear. The supernatant was then discarded and the beads were mixed with 200 μl of freshly prepared 80% ethanol and incubated on the magnetic plate for at least 30 seconds at room temperature. The supernatant was carefully removed and the beads were washed twice with ethanol again. The resulting beads were allowed to stand at room temperature for 5 minutes or until the pellet appeared dry. The beads were then re-suspended in 51 μl of re-suspension buffer, mixed by pipetting and incubated at room temperature for another 2 minutes. The tube was placed again on the magnetic stand for 2 minutes or until the supernatant was completely clear. From this, 50 μl of the clear supernatant was transferred to a fresh tube. To this clear supernatant, 40 μl of AMPure XP beads was added, incubated on a magnetic stand for 5 minutes at room temperature or until the supernatant was completely clear. The beads were washed again with 200 μl of freshly prepared ethanol. After the second wash the supernatant was removed and the beads were allowed to stand at room temperature for 5 minutes or until the pellet appeared dry. The resulting dry beads were then re-suspended in 35 μl re-suspension buffer, incubated at room temperature for 2 minutes and then placed again on the magnetic stand for another 5 minutes or until the supernatant was completely clear. From this 34 μl of supernatant was transferred to a fresh tube for further processing such as amplification.

### PCR Amplification

34 μl of ligated DNA was then mixed with 12 μl of NEXTFlex™ PCR master mix, 2 μl of NEXTFlex qRNA-Seg™ universal forward primer, NEXTFlex qRNA-Seg™ barcoded primer to form a 50 μl volume mix, mixed well and amplified for 15 PCR cycles by incubating the tubes in the following reaction of 2 minutes at 98° C., 30 seconds at 98° C., 30 seconds at 65° C., 60 seconds at 72° C. and 4 minutes at 72° C.

### Suppression of Adaptor Dimers

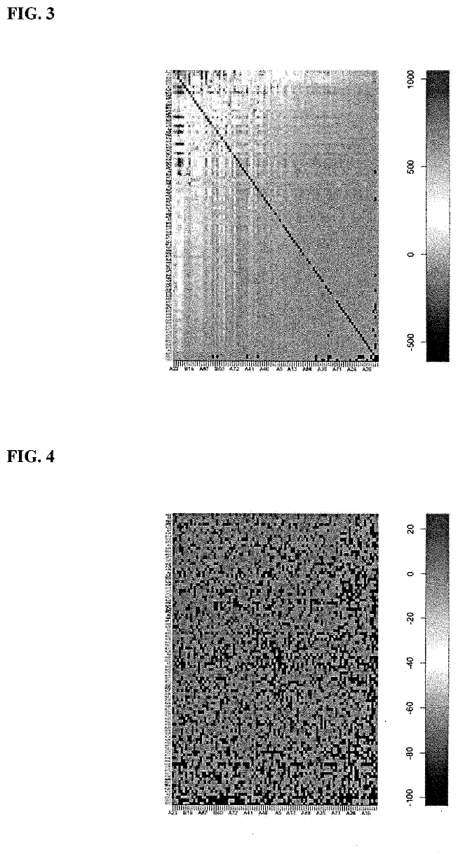

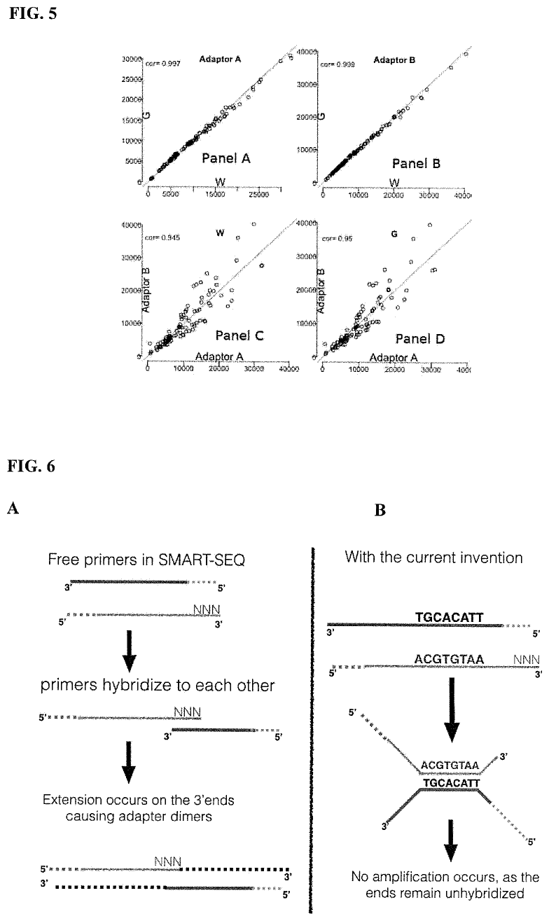

The library prepared according to the method described above was then subjected to sequencing. The resulting sequencing data was further analyzed for the presence of adaptor dimers and the adaptor dimer data was then plotted to show deviations from the mean, or expected values if the barcode pairs randomly assorted, as shown in FIGS. 3 and 4.

A striking feature of the data shown in FIG. 3, is the lack of adaptor dimer pairs or suppression of adaptor dimer formation when the adaptor ends have the same barcode on both sides (as shown in FIG. 1D). Surprisingly, the data in FIG. 3 also revealed that the diagonal elements (the inserts with adaptors on either side with complementary ends), are not suppressed when there is an insert between the adaptors, suggesting this method works well in selectively suppressing adaptor dimers. We believe this is due to a hairpin formation which potentially inhibits amplification of the insert (FIG. 1E). This gives us an easy method of suppressing adaptor-dimers by using ends that are complementary to each other.

The experiment was repeated to show that the data are consistent between two different experiments, suggesting that the results are reproducible as evident from FIG. 4.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “substantially” is used in the specification or the claims, it is intended to take into consideration the degree of precision available or prudent in manufacturing. To the extent that the term “operably connected” is used in the specification or the claims, it is intended to mean that the identified components are connected in a way to perform a designated function. As used in the specification and the claims, the singular forms “a,” “an,” and “the” include the plural. Finally, where the term “about” is used in conjunction with a number, it is intended to include ±10% of the number. In other words, “about 10” may mean from 9 to 11.

As stated above, while the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art, having the benefit of the present application. Therefore, the application, in its broader aspects, is not limited to the specific details, illustrative examples shown, or any apparatus referred to. Departures may be made from such details, examples, and apparatuses without departing from the spirit or scope of the general inventive concept.

1. A method for suppressing or preventing adaptor dimer formation comprising the steps of:
(i) providing a target polynucleotide with a 5′ and a 3′ end,
(ii) providing at least two adaptors with ends having nucleotide sequence that is complementary to each other, and
(iii) ligating the adaptor ends to the target polynucleotide to form a ligation product.

2. The method of claim 1, wherein the two adaptors are double stranded DNA adaptors.

3. The method of claim 1, wherein the two adaptors are single stranded RNA adaptors.

4. The method of claim 1, wherein the two adaptors are single stranded DNA adaptors.

5. The method of claim 1, wherein one of the two adaptors is a single-stranded RNA adaptor and the other is a single-stranded DNA adaptor.

6. The method of claim 1, wherein at least one of the two adaptors can be a hybrid of DNA and RNA.

7. The method of claim 1, wherein the target polynucleotide is a double stranded DNA or complementary DNA.

8. The method of claim 1, wherein the ligation product is the target polynucleotide with the adaptor ends having a complementary sequence flanking on each end of the target.

9. The method of claim 1, wherein the adaptors are at least a 4-mer sequence.

10. The method of claim 1, wherein the adaptors are at least an 8-mer sequence.

11. The method of claim 1, wherein the method suppresses the adaptor dimer formation by more than about 90%.

12. The method of claim 1, further comprising a double stranded DNA ligase.

13. The method of claim 1 requires no addition of a hairpin oligonucleotide to the ligation reaction.

14. A method of preparing a library of nucleic acid sequences comprising the steps of:
(i) providing at least two adaptors with ends having nucleotide sequence complementary to each other,
(ii) contacting the adaptor with a target nucleic acid sequences having a 5′ and a 3′ end, and
(iii) ligating the adaptor having the complementary sequence to the 5′ and 3′ ends of the target nucleic acid sequence using a double stranded DNA ligase or single stranded RNA ligase.

15. The method of claim 14, wherein the adaptor ends flanking the target nucleic acid sequence is configured to suppress the formation or abundance of adaptor dimers.

16. The method of claim 14, wherein the adaptors are double stranded DNA or a single stranded RNA.

17. The method of claim 14, wherein the target nucleic acid sequence is a double stranded DNA or a complementary DNA (cDNA).

18. A method for suppressing or preventing adaptor dimer formation in SMART sequencing comprising the steps of:
(i) providing a target polynucleotide with a 5′ and a 3′ end,
(ii) providing at least two adaptors with ends having nucleotide sequence that is complementary to each other, and
(iii) adding the adaptor ends to the target polynucleotide in a ligase free reaction.

19. The method of claim 18, wherein the target polynucleotide is a complementary DNA.

20. The method of claim 18, further comprising addition of reverse transcriptase to facilitate the synthesis of complementary DNA.

21. The method of claim 18, further comprising addition of a first strand synthesis primer and a template switching primer.

22. A kit for suppressing adaptor dimer formation comprising: at least two oligonucleotide adaptors having nucleotide sequence that is complementary to each other.

23. The kit of claim 22, wherein the adaptors are single stranded RNA or double stranded DNA.

24. The kit of claim 22, wherein the adaptors are at least a 4-mer sequence.

25. The kit of claim 22 further comprising an enzyme selected from the group consisting of ligase or polymerase.
